(19) United States
(12) Patent Application Publication
(10) Pub. No.: US 2024/0123254 A1
(43) Pub. Date: Apr. 18, 2024

(54) WEARABLE LIGHT THERAPY DEVICE, SYSTEM AND METHOD FOR THE SAME

(71) Applicant: WIDIC Industries LLC, Los Angeles, CA (US)

(72) Inventor: Sarah Nikolovska, Los Angeles, CA (US)

(21) Appl. No.: 18/457,940

(22) Filed: Aug. 29, 2023

Related U.S. Application Data
(60) Provisional application No. 63/379,556, filed on Oct. 14, 2022.

(51) Int. Cl. A61N 5/06; A61B 5/33

(52) U.S. Cl. CPC A61N 5/0622; A61B 5/33; A61N 2005/0626; A61N 2005/0647; A61N 2005/0651; A61N 2005/0663

(57) ABSTRACT

Embodiments include a wearable device comprising a housing configured for coupling to an outer ear of a user; a light source disposed in the housing to direct a light output towards a region at or near the outer ear innervated by a vagus nerve branch; and at least one processor configured to transmit, to the light source, a control signal for controlling emission of the light output. Embodiments also include a method comprising transmitting, to a light source, a first control signal configured to initiate emission of a light output towards a region at or near the outer ear innervated by a vagus nerve branch; receiving biometric data from a biometric sensor while the light output is directed towards the region; determining an abnormal user response based on the received data; and responsive thereto, transmitting a second control signal to the light source to stop emission of the light output.

### CROSS-REFERENCE

This application claims priority to U.S. Provisional Patent App. No. 63/379,556, filed on Oct. 14, 2022, the contents of which are incorporated herein by reference in their entirety.

### BACKGROUND

Light-based therapy, or light therapy, has been used to treat various ailments in the human body, including, but not limited to, pain or inflammation, acne, hair loss, obesity, aging, wound healing, and more. One form of light therapy, known as photobiomodulation therapy, uses non-ionizing forms of light sources, such as low level lasers, light emitting diodes (“LED”), and broad-band light, in the visible and near infrared (NIR) spectrum to elicit non-thermal physical and chemical changes within the cells of the human body, which may result in beneficial changes to the tissue, system, or throughout the body. These effects are attributed to the ability of certain structures in the human body, such as proteins in a cell, to become “activated” upon absorbing light. The most specialized light-sensitive structures in the body are located in the eye and are associated with sight and the synchronizing of the body's functions to the external environment (e.g., during daytime, nighttime, etc.). However, light-responsive proteins are located in many other areas of the body as well, such as the skin, the central nervous system, peripheral nerves, and blood vessels. Given that different wavelengths of light penetrate the skin to different depths, different colors of light can be used to target different structures in these areas of the body, such as, for example, specific structures in the brain with potent NIR lasers.

Relatedly, laser or light acupuncture uses low-intensity, non-thermal laser irradiation to stimulate traditional acupuncture points for treatment or relief of numerous ailments, including, but not limited to, acute inflammation, visceral, and neuropathic pain. This method involves placing laser pens, or laser needles, in direct contact with the skin (without puncturing it), positioning the laser beam vertically to the skin surface to reduce reflection and optimize energy density, and selecting a color for the laser based on the desired depth of penetration. For example, NIR lasers are used for deep pain treatment and wound healing, while green lasers are used to stimulate more shallow acupuncture points (such as, e.g., those found in the ear).

A few wearable devices have been developed for applying specific forms of light therapy to a user. For example, U.S. Pat. No. 9,364,683 describes a portable electronic device configured to be worn in the user's ear and comprising an LED for directing optical radiation (i.e. laser light) into the ear canal, in order to stimulate intracranial nerve tissue of the user's brain. The owners of that patent also own U.S. Pat. No. 9,258,642, which adds an audio transducer to the earpiece, so that the user can play audio, either separately or in conjunction with receiving optical radiation via the ear canal. Others have developed a helmet lined with LEDs that applies red and near-infrared light to the scalp and ears of the user with the goal of improving thinking and memory, specifically in war veterans.

Other existing wearable devices use electric pulses, instead of light, to stimulate nerves for therapeutic purposes. For example, U.S. Pat. Nos. 11,241,574 and 11,235,156 describe earpiece-like devices configured to deliver electrical stimulation to an auricular branch of the vagus nerve in conjunction with an audio therapy regimen, also delivered via the earpiece, to help relieve insomnia or anxiety. The earpieces include electrodes for applying an electric pulse to the ear canal, concha, and/or tragus portions of the wearer's outer ear. The earpieces also include sensors for determining the occurrence of a physiological event (e.g., an anxiety attack or migraine headache) and stimulating the auricular vagus nerve based on said determination.

One major drawback of electrical stimulation is that it can be uncomfortable, and even painful, because the amplitude of the pulses must be set as high as the user can tolerate in order to maximize the desired effect of the electrical stimulation. As a result, the electric pulses may cause irritation or burns to the surface of the skin.

Accordingly, there is still a need in the art for an improved non-invasive therapy device that can provide physiological benefits without causing discomfort or pain.

### SUMMARY

The invention is intended to solve the above-noted problems by providing systems and methods that are designed to, among other things, provide light therapy to a branch of the vagus nerve located in the auricular or peri-auricular region of a person, for example, using a device that is configured to be worn in, on, or near the ear, in order to provide physiological benefits throughout the user's body.

For example, one embodiment provides a wearable device, comprising: a housing configured for coupling to an outer ear of a user; a light source disposed in the housing and configured to direct a light output towards a region of the outer ear that is innervated by a branch of a vagus nerve of the user; and at least one processor disposed in the housing and communicatively coupled to the light source, the at least one processor configured to transmit, to the light source, a control signal for controlling emission of the light output by the light source.

Another exemplary embodiment provides a system, comprising: a first housing configured for coupling to a first outer ear of a user, the first housing comprising a first light source configured to direct a first light output towards a first region at or near the first outer ear, the first region innervated by a first branch of a vagus nerve of the user; a second housing configured for coupling to a second outer ear of the user, the second housing comprising a second light source configured to direct a second light output towards a second region at or near the second outer ear, the second region innervated by a second branch of the vagus nerve; and one or more processors configured to provide a first control signal to the first light source to control emission of the first light output, and a second control signal to the second light source to control emission of the second light output.

Another exemplary embodiment provides a method performed by at least one processor in communication with a light source and a biometric sensor, the method comprising: transmitting, to the light source, a first control signal configured to initiate emission of a light output towards a region at or near an outer ear of a user, the region innervated by a branch of a vagus nerve of the user; receiving, from the biometric sensor, biometric data of the user while the light output is directed towards the region; determining an abnormal user response based on the received biometric data; and responsive to the abnormal user response, transmitting, to the light source, a second control signal configured to stop emission of the light output.

Yet another exemplary embodiment provides a method performed by at least one processor in communication with a light source and a biometric sensor, the method comprising: receiving, from the biometric sensor, biometric data of a user; determining an abnormal user response based on the received biometric data; and responsive to the abnormal user response, transmitting, to the light source, a first control signal configured to initiate emission of a light output towards a region at or near an outer ear of the user, the region innervated by a branch of a vagus nerve of the user.

These and other embodiments, and various permutations and aspects, will become apparent and be more fully understood from the following detailed description and accompanying drawings, which set forth illustrative embodiments that are indicative of the various ways in which the principles of the invention may be employed.

### BRIEF DESCRIPTION OF THE DRAWINGS

FIGS. 1A and 1B are front and back views, respectively, of an external portion of an exemplary human ear.

FIG. 2 is a block diagram of an exemplary light therapy system, in accordance with embodiments.

FIG. 3 is a perspective view of an exemplary wearable light therapy device, in accordance with embodiments.

FIG. 4 is a front view of an ear cup of the wearable light therapy device shown in FIG. 3, in accordance with embodiments.

FIG. 5 is a partial top view of only the earcups of the wearable light therapy device of FIG. 3, shown relative to a human head, in accordance with embodiments.

FIGS. 6A-6C are first and second side views and a front view, respectively, of another exemplary wearable light therapy device, in accordance with embodiments.

FIG. 7 is a back view of the wearable light therapy device of FIGS. 6A-6C, worn on an ear of a user, in accordance with embodiments.

FIG. 8 is a flow diagram of an exemplary method of providing light therapy, in accordance with embodiments.

### DETAILED DESCRIPTION OF EXAMPLE EMBODIMENTS

The systems, methods, and devices described herein can be used to perform auricular or periauricular chromatherapy, a form of light therapy that involves applying LED or laser light to certain regions of or near a user's ear while the user recalls a particularly emotionally-charged or traumatic event, in order to alleviate symptoms of anxiety and depression associated with the event, or more generally, to treat anxiety disorders (e.g., phobias, panic attacks, etc.). The process draws upon the ability of light to stimulate activity within the nervous system, specifically within structures associated with emotion and physical sensation.

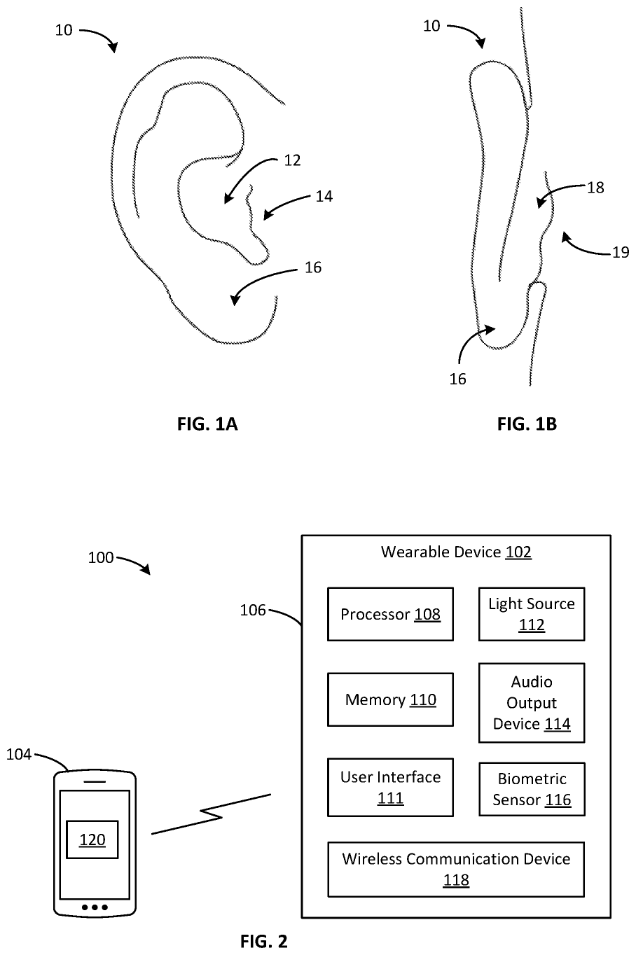

FIGS. 1A and 1B show an exemplary outer ear 10, or the portion of the human ear that is located external to the human body and outside the ear canal (not shown). The outer ear 10, also known as the “auricle” or “auricular region” of the ear, is made of cartilage and skin, and includes a concha cavity or area 12 (or “concha”), a tragus area 14 (or “tragus”), an ear lobe 16 (or “lobule”), a back or rear surface 18 of the ear near the scalp, and other areas of the ear, such as, e.g., helix and antihelix portions towards a top of the outer ear 10 and an antitragus portion below the concha area 12 (not shown). FIG. 1B also shows a scalp area 19 near the back surface 18 of the outer ear 10. As will be appreciated, a branch of the vagus nerve passes through this scalp area 19 as it extends into the outer ear 10, for example, towards the concha area 12, tragus area 14, and ear lobe 16.

Studies have shown that projecting certain colors of light towards specific regions of the human ear may have different beneficial effects. For example, projecting yellow light (e.g., 560-590 nanometers (nm)) towards the ear lobe 16, or other region of the ear associated with the limbic system (i.e. the part of the brain that generates emotion), during recall of a memory, can break the link between the memory and the emotion generated thereby, thus neutralizing the memory. As another example, projecting blue light (e.g., 450-490 nm) over the concha area 12, or other region of the ear associated with the viscera, may reduce and/or eliminate the physical symptoms of stress and anxiety associated with a particular memory. It has also been demonstrated that full body expose to blue light increases production of beta endorphins and decreases blood pressure through the release of nitric oxide from the skin.

As will be appreciated, the vagus nerve is the major parasympathetic nerve, extending from the base of the brain throughout the human body, including up into the ears and down into the lower intestines. Generally speaking, stimulating the vagus nerve has been shown to bring the body into a relaxation state that can help the human body heal naturally, with the related physiological effects extending throughout the human body. For example, stimulation of the vagus nerve can modulate, or have beneficial effect on, tinnitus, hormones, hiccups, digestion or gut healing, glucose levels and/or other physiological conditions related to metabolic disease (e.g., diabetes), as well as inflammation, including autoimmune disease (e.g., rheumatoid arthritis), inflammatory brain disease (e.g., Alzheimer's, Parkinson's, etc.), IBS or Chrone's disease, and other inflammatory diseases. Stimulating the vagus nerve can also modulate brain chemistry to allow for improvements in mood (e.g., depression), sleep (e.g., insomnia), alertness, learning, anxiety, stroke recovery (e.g., nerve regeneration), and more, as well as treatment of pain, post-traumatic stress disorder (PTSD), drug resistant epilepsy, opioid dependency, and other conditions. In addition, stimulating the vagus nerve can have beneficial cardiovascular effects, such as, but not limited to, regulation of heart rate, high blood pressure, arrhythmia, and more, as well as improvement of cardiac remodeling. Vagus nerve stimulation has also been shown to reduce the symptoms of visually induced motion sickness and simulator adaptation syndrome.

The inventor has discovered that stimulating or activating the vagus nerve by directing a light output (e.g., LED light or laser light) towards certain regions of, or near, the outer ear 10 that are innervated by, or include, a branch of the vagus nerve enables treatment of physiological conditions throughout the human body (e.g., those listed above) in a more comfortable and non-invasive manner. For example, targeting the concha 12 of the outer ear 10, the tragus 14 of the outer ear 10, the back surface 18 of the outer ear 10, and/or the nearby scalp area 19 with the light output can activate the vagus nerve and thereby, decrease a heart rate of the user, decrease respiratory rate, increase heart rate variability, induce relaxation, and/or otherwise provide a physiological release, without causing pain or discomfort to the user. The inventor has also discovered that certain wavelengths of light are more effective in this regard than others, such as, for example, violet to blue light (e.g., light at a wavelength of about 420 nanometers (nm) to about 470 nm) or green light (e.g., light at a wavelength of about 500 nm to about 565 nm). Moreover, the inventor has discovered that certain wavelengths are more effective when targeting certain regions of the outer ear 10. For example, violet to blue light (e.g., about 430 nm) may be more effective at inducing relaxation when directed towards the concha area 12 of the outer ear 10 or the scalp area 19 behind the outer ear 10, while green light (e.g., about 510 nm) may provide better results when directed towards the back surface 18 of the outer ear 10. As another example, yellow light (e.g., about 550 nm) may be more effective when directed towards the tragus area 14 of the outer ear 10.

Referring now to FIG. 2, shown is an exemplary light therapy system 100 for implementing the techniques described herein, in accordance with embodiments. In particular, the light therapy system 100 comprises a wearable light therapy device 102 configured to direct light towards a vagus nerve of a user, or wearer, of the device 102. The system 100 also comprises a remote device 104 communicatively coupled to the wearable device 102 for controlling operation of the wearable light therapy device 102 (also referred to herein as “wearable device 102”), including emission of the light directed towards the user.

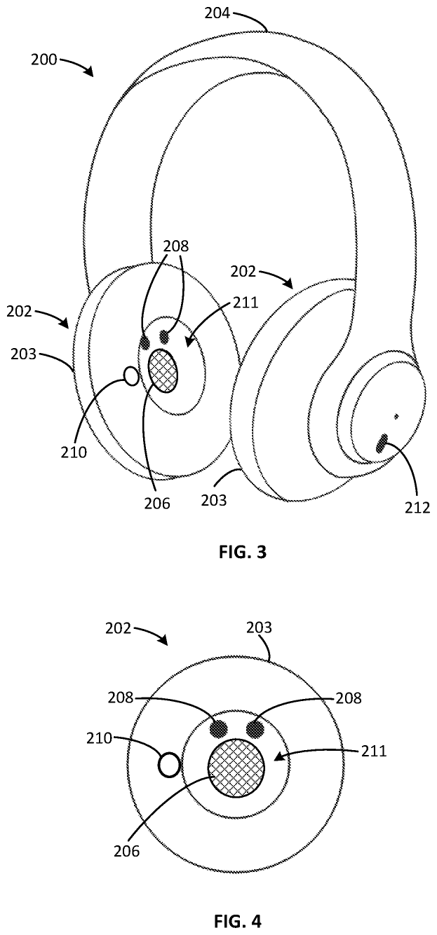

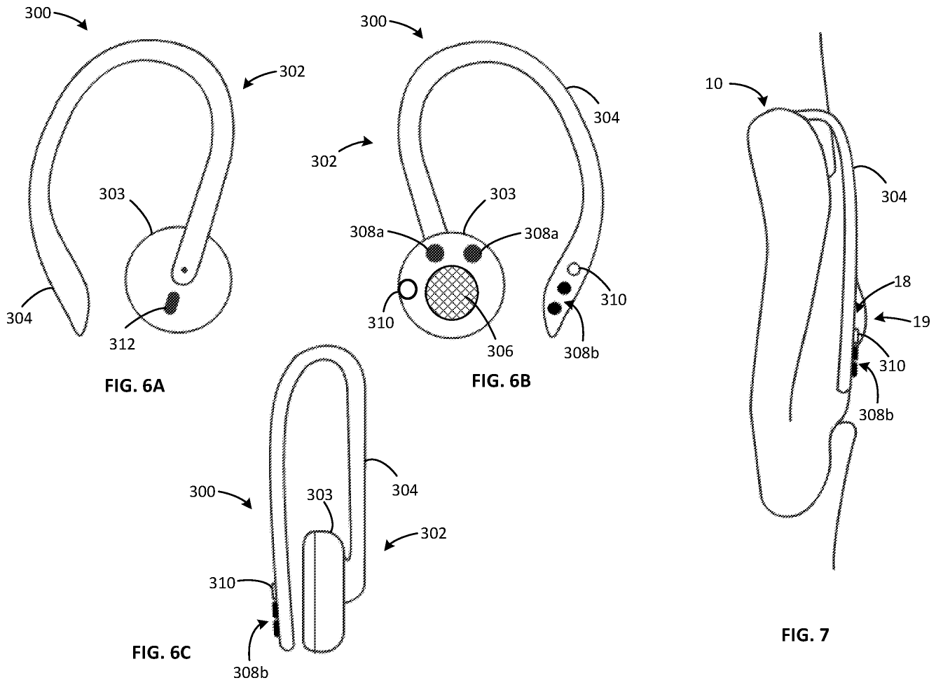

According to embodiments, the wearable device 102 may be any type of personal device or apparatus with a housing 106 configured to be worn on a body of the user, or otherwise attached to the user, at or adjacent to an area of the body that is innervated by the vagus nerve. In some embodiments, the wearable device 102 may be a headset, headphones, an earpiece, or any other personal listening device (e.g., headphones 200 shown in FIG. 3, or earbud 300 shown in FIGS. 6A-6C), a virtual reality (VR) headset, or any other headpiece wherein the housing 106 is configured to be worn on or over one or more ears of the user in order to target a branch of the vagus nerve that extends into or near the outer ear 10 (such as, e.g., the branch of the vagus nerve that extends through or near the concha area 12 shown in FIG. 1A). In other embodiments, the wearable device 102 may be a headband or other apparatus wherein the housing 106 is configured to be coupled to the user's head adjacent the ears of the user in order to target the branch of the vagus nerve that extends through the back of the outer ear 10 (such as, e.g., the branch of the vagus nerve that extends through the scalp area 19 or the back ear region 18 shown in FIG. 1B). In one exemplary embodiment, the wearable device 102 may be an adhesive patch or sticker, a tag, or other removable unitary device wherein the housing 106 is configured to be attached to the scalp area 19 of the user, or other skin surface near the back of the outer ear 10 that is suitable for targeting the branch of the vagus nerve that extends into the outer ear 10. In still other embodiments, the wearable device 102 may be configured to be worn on or near one or more other areas of the body that are innervated by other branches of the vagus nerve, such as, e.g., the neck, chest, abdomen, etc. In such cases, for example, the housing 106 may be coupled to, integrated into, or included on a band, belt, strap, clip, or other article that can be secured to, or around, the user's body and/or clothing worn by the user.

As shown in FIG. 2, the wearable device 102 further comprises a plurality of electronic components, including processor 108, memory 110, user interface 111, light source 112, audio output device 114, biometric sensor 116, and wireless communication device 118. Housing 106 can be configured to house or encase each of these electronic components, as illustrated. In some embodiments, the wearable device 102 may include multiple wearable housings 106 (such as, e.g., two earpieces 206 shown in FIG. 3) in order to target multiple areas of the user's body at once (e.g., both ears of the user). In such cases, the multiple housings 106 may be substantially similar to each other in terms of structure, configuration, and/or operation. For example, each housing 106 may comprise the electronic components shown in FIG. 2, or a subset of the same. In some cases, the housings 106 may include a first housing that has been designated as a master device and a second housing that has been designated as a slave device, so that incoming signals from the remote device 104 need only be transmitted to the first housing, which then relays the signals to the second housing, and vice versa.

Processor 108 can include one or more of a baseband processor, an audio processor, a data processor, a central processing unit, a microprocessor, a microcontroller, a programmable logic array, an application-specific integrated circuit, a logic device, or other electronic device for processing, inputting, outputting, manipulating, storing, or retrieving data. Processor 108 can be configured to execute software stored in memory 110 of the wearable device 102. Each of the memory 110, user interface 111, light source 112, audio output device 114, biometric sensor 116, and wireless communication device 118 can be communicatively coupled to the processor 108.

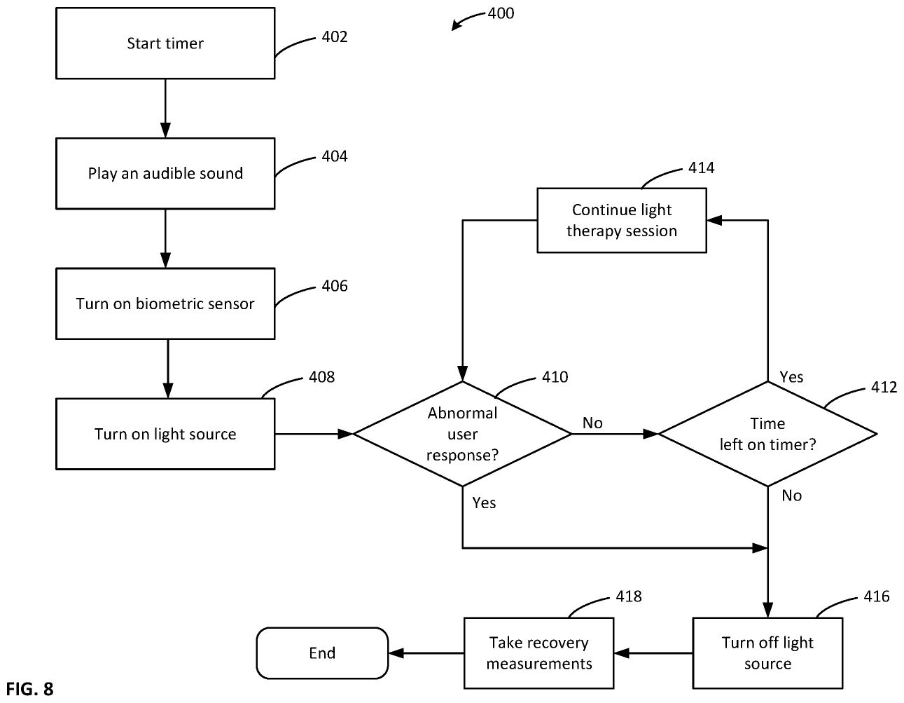

Memory 110 can comprise one or more of a data storage device, an electronic memory, a nonvolatile random access memory (e.g., RAM), flip-flops, a non-transitory computer-writable or computer-readable storage medium, a magnetic or optical data storage device, or other electronic device for storing, retrieving, reading, or writing data. Memory 110 can store one or more computer program modules, computer executable instructions, or other software for execution by the one or more processors 108. For example, in embodiments, memory 110 may store a software application (not shown), which, when executed by processor 110, causes processor 110 to implement the light-based therapy techniques described herein and/or a method described herein (such as, for example, method 400 shown in FIG. 8).

User interface 111 can be used by the user to control operation and/or adjust one or more settings of the wearable device 102. For example, the user interface 111 can be configured to enable the user to turn the wearable device 102 on or off, initiate communication with the remote device 104, start or stop emission of a light output from the light source 112, start or stop an audible content played by the audio output device 114, start or stop operation of the biometric sensor 116, adjust a duration and/or intensity of the light output from the light source 112, adjust a volume of the audible content played by the audio output device 114, adjust a setting of the biometric sensor 116, and more. In various embodiments, the user interface 111 may include one or more user input devices, such as, for example, a button, slider, switch, touchscreen, touchpad, joystick, microphone, keyboard, keypad, and/or any other device capable of receiving an input from the user.

Light source 112 can comprise at least one light emitting diode (“LED”), laser diode, or other type of electronic device capable of emitting or directing a light output, or beam of light, towards a selected region, or targeted area, of the user's body. In various embodiments, the light source 112 can be configured to direct a light output towards a region of the outer ear 10 that is innervated by, or includes, an auricular vagus nerve of the user (or a branch of the vagus nerve that extends into an ear of the user). For example, the innervated region of the outer ear 10 may include the concha area 12, the tragus area 14, the back surface 18 of the outer ear 10, or any combination thereof. In some embodiments, the light source 112 can be configured to direct the light output towards a region of the scalp near the outer ear 10 that is innervated by a branch of the vagus nerve outside the outer ear 10 (e.g., the same branch that later extends into the outer ear 10). For example, the innervated region of the scalp may include the scalp area 19 at the back of the outer ear 10, as shown in FIG. 1B. In other embodiments, the light source 112 may be configured to direct the light output towards another region of the user's body that is innervated by the vagus nerve, such as, e.g., the neck or abdomen.

In various embodiments, the light source 112 can be configured to continuously emit the light output for the duration of the therapy session, for a preset amount of time (e.g., twenty minutes), or other time period. In some embodiments, the light source 112 may be configured to emit the light output periodically, for example, in bursts or flashes, during the therapy session. In either case, the processor 108 can be configured to send, to the light source 112, a first control signal at the start of a therapy session to turn on the light source 112 or otherwise begin emitting the light output, and a second control signal at the end of the therapy session to turn off the light source 112 or otherwise stop emission of the light output. In some embodiments, in addition to duration of the light output, the processor 108 can also be configured to control an intensity of the light output, for example, by sending a third control signal to the light source 112 for increasing or decreasing the light intensity.

In some cases, the processor 108 may be configured to control the light intensity and/or duration of the light output based on user inputs received via the user interface 111. In other cases, the processor 108 may be configured to automatically control the light intensity and/or duration based on data received from the remote device 104 and/or other components of the wearable device 102, such as, e.g., biometric data from the biometric sensor 116. For example, in various embodiments, the processor 108 can be configured to automatically turn off the light source 112 if a negative response (e.g., vagus withdrawal) is detected based on biometric data obtained by the biometric sensor 116 during the light therapy session.

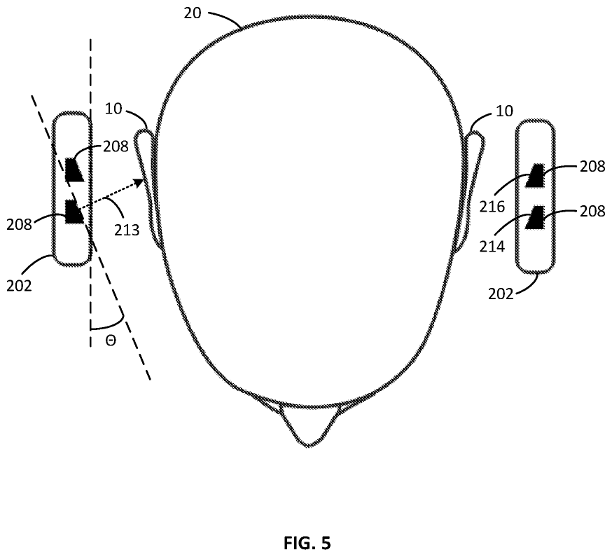

The light source 112 can be configured to direct the light output substantially perpendicular to the selected region, or more specifically, to a skin surface of that region, to minimize reflections or other distortions as the light penetrates the skin surface and travels to the vagus nerve. In some cases, the light source 112 may be placed within the housing 106 at a fixed angle or position so that, once the housing 106 is coupled to or placed on the target area, the light beam output by the light source 112 is directed or angled substantially perpendicular to the target area (e.g., as shown in FIG. 5). In other cases, the light source 112 may be movable or otherwise configured to adjust an angle of incidence of the light output on the target area. For example, the light source 112 may be mounted on a swivel, rotary, or other movable mechanism for enabling angular or positional adjustments. In such cases, movement of the light source 112 may be manually controlled (e.g., by the user) or automatic implemented (e.g., using processor 108 executing software stored in the memory 110).

In some cases, the housing 106 may include multiple light sources 112 to enable the wearable device 100 to target different areas of the user's body, as needed. The multiple light sources 112 may be operated simultaneously, consecutively (e.g., alternating between the light sources one by one), or in any other manner. As an example, the housing 106 may include a first light source 112 directed towards a front surface of the outer ear 10 (e.g., the concha 12 or the tragus 14) and a second light source 112 directed towards the back surface 18 of the outer ear 10 and/or the scalp area 19 (e.g., as shown in FIGS. 6B and 7). In some cases, the light source 112 (or each light source 112) may include a plurality of LEDs or other light elements in order to increase a light field of the light output that is directed towards the selected region. In other cases, the light source 112 may include a plurality of LEDs or other light elements in order to target multiple innervated regions that are adjacent to each other (e.g., the concha 12 and the tragus 14 of the outer ear 10, as shown in FIG. 1A), or other different areas of or near the outer ear 10 (e.g., front of the outer ear 10 and back of the outer ear 10).

According to embodiments, the light source 112 can be configured to emit light at a specific wavelength of visible light. For example, the light source 112 may be configured to emit violet light, blue light, or any other light having a wavelength selected from a range of about 420 nanometers (nm) to about 470 nm (referred to herein as “violet to blue light”). As another example, the light source 112 may be configured to emit green light, yellow light, or any other light having a wavelength selected from a range of about 500 nm to about 565 nm (referred to herein as “green to yellow light”). In one embodiment, the light source 112 includes a violet-blue LED with a peak wavelength of 420 to 440 nm. In another embodiment, the light source 112 includes a green LED with a peak wavelength of, or about, 510 nm. In another embodiment, the light source 112 includes a yellow LED with a peak wavelength of, or about, 550 nm. Other colors of visible light may also be used in accordance with the techniques described herein. In various embodiments, the color of the light source 112 may be selected depending on the target area of the user's body, such as, for example, a thickness of the skin at that area, a distance from the skin surface to the vagus nerve, any obstacles below the skin surface along the path of the light output (e.g., internal organs, etc.), and/or other characteristics that are associated with or influence an efficacy of the light output.

Audio output device 114 can be any type of audio listening device configured to generate an audible sound based on an audio signal received from the processor 108. For example, the audio output device 114 may include one or more speaker transducers or other loudspeakers, or any other audio playback device. The audio signals provided to the audio output device 114 may include any type of audio content including, for example, musical content (e.g., instrumental music and/or vocal music), speech content (e.g., spoken word or other human speech), or any combination thereof. In some cases, the audio signals represent standalone audio content, such as, for example, a musical piece, a podcast, or other audio recording. In other cases, the audio signals are part of audio-visual content, such as, for example, a video game, a movie, or other video-based application.

In some embodiments, the processor 108 can be configured to simultaneously, or nearly simultaneously, provide both the light output and the audible sound to the user. For example, the processor 108 can be configured to instruct the audio output device 114 to begin playback of an audio signal and, at the same time or nearly the same time, instruct the light source 112 to turn on or begin emitting the light output. In some cases, the audio output device 114 may be configured to continuously, or nearly continuously, play audio during the light therapy session, or while the light output is being emitting. In such cases, the audio signal may include therapeutic audio content associated with inducing relaxation, relieving anxiety, and/or providing other therapeutic benefits during the light therapy session. For example, the therapeutic audio content may include audio related to eye movement desensitization and reprocessing (“EMDR”) therapy (such as, e.g., verbal instructions to identify an image associated with a traumatic event, also known as trauma script exposure or imaginal exposure), or any other bilateral tonal or music content that alternates playback between the left and right ears. Other types of therapeutic audio content are also contemplated, including, for example, audio associated with Prolonged Exposure Therapy, audio associated with Cognitive Behavioral Therapy, music for inducing relaxation, neutral audio background sounds (e.g., white noise, etc.), and more.

In some cases, the audio output device 114 may be configured to play instructional audio content at certain times during the therapy session, such as, for example, before, during, or after the session, at the start or end of the session, etc., in addition to, or instead of, the therapeutic audio content. In such cases, the audio signal provided to the audio output device 114 may include audio cues for informing the user of certain events or otherwise guiding the user during the therapy session. For example, the audio cues may be configured to signal or indicate when the light source 112 is turning on or off, when a timer associated with light therapy application is starting or stopping, when abnormal biometric readings are detected, and more. As another example, the audio cues may be configured to instruct the user to close their eyes, adjust their body position, clear their mind, draw attention to areas of tension or pain in the body, become aware of any intrusive thoughts or ongoing emotional conflicts present in their lives, and more. The audio cues may be audibly output by the audio output device 114 before, during, or after emission of the light output by the light source 112, depending on the type of audio cue. For example, the processor 108 may be configured to send an initial audio signal to the audio output device 114 for playback before turning on the light source 112.

The biometric sensor 116 can be any type of photosensor, transducer, or other device capable of detecting or capturing a relevant biometric trait of the user and converting that biometric trait into an electrical signal. In various embodiments, the biometric sensor 116 can be used to monitor the user's response to the light therapy and automatically control operation of the light source 112 if the user exhibits a negative response, such as, for example, signs of vagus withdrawal or vagal overstimulation (e.g., rapid increase in heart rate, increased breathing, dizziness, clamminess, or other signs of high sympathetic tone) due to a sensitivity to the light output, or other abnormal vagal response. For example, the biometric sensor 116 can be configured to obtain biometric data of the user during emission of the light output towards the user and send the obtained biometric data to the processor 108, and the processor 108 can be configured to control operation of the light source 112 based on the received data. In some cases, the processor 108 may be configured to stop emission of the light output (or turn off the light source 112) if the received biometric data falls below a predetermined threshold or baseline measure, or otherwise indicates an abnormal or negative user response. In some cases, the processor 108 may be configured to automatically adjust a setting of the light output (e.g., intensity, duration, etc.) based on the biometric data, for example, in order to control or temper the user's response before overstimulation occurs. In some embodiments, the biometric sensor 116 can be configured to collect or detect biometric data continuously, or nearly continuously, such that a stream of biometric data is provided to the processor 108 for analysis. The data collected by the biometric sensor 116 may be stored in memory 110 or other memory unit of the wearable device 102, and/or in a memory of the remote device 104, for future processing and/or comparison.

In various embodiments, the biometric data obtained by the biometric sensor 116 may include information associated with an electrical activity of a heart of the user, such as, for example, heart rate, heart rate variability (“HRV”), heart rate intervals, or other electrical signal associated with the heart, and the biometric sensor 116 may be a heart rate monitor, a heart rate variability monitor, or other device capable of monitoring the electrical activity of the heart, respectively. As an example, in embodiments where the biometric sensor 116 is an HRV monitor, a negative user response, or abnormal HRV reading, may be detected if there is a sustained decrease in HRV, as compared to a threshold value or baseline measure, over a select length of time. Conversely, a normal user response or HRV reading may be detected when the HRV increases or remains the same over the select length of time. In other embodiments, the biometric data may include information related to other biometric traits that may indicate a negative response of the user, such as, for example, a sudden drop in blood pressure followed by a sharp rise of the same (e.g., a rapid sympathetic recovery).

In various embodiments, the wireless communication device 118 may be used to enable wireless communication between the wearable device 102 and the remote device 104 or other component of the system 100. For example, the wireless communication device 118 can be configured to receive control signals (e.g., the control signal for controlling emission of the light output by the light source 112), audio signals (e.g., the audio signal for generating the audible sound using the audio output device 114), or other data from the remote device 104. As another example, the wireless communication device 118 can be configured to send data, (e.g., the biometric data obtained by the biometric sensor 116) to the remote device 104. In embodiments that include multiple housings 106, the wireless communication device 118 may also be configured to enable communication with another housing 106 of the wearable device 102, for example, in order to relay control signals received at a first housing to a second housing, or vice versa.

The wireless communication device 118 may be configured to transmit and/or receive data using short-range wireless communications technology, such as, e.g., Bluetooth®, radio-frequency identification (“RFID”), near field communication (“NFC”), etc., wide area network communications technology, such as, e.g., WiFi, Zigbee, etc., or any other suitable wireless communications technology, such as, e.g., cellular, etc. Though not shown, the wireless communication device 118 may include antennas, modems, transceivers, receivers, transmitters, and/or other wireless communication circuitry suitable for carrying out the wireless communications described herein.

The remote device 104 may be a computing device, control unit, or any other type of electronic device capable of interfacing with the wearable device 102 in order to control operation of the wearable device 102. In some embodiments, the remote device 104 may be a mobile communication device (e.g., cellphone, smartphone, etc.) or other type of mobile computing device (e.g., tablet, PDA, etc.), or a smartwatch or other type of wearable mobile device. In other embodiments, the remote device 104 may be a personal computer (e.g., laptop, desktop, etc.). In still other embodiments, the remote device 104 may be a standalone electronic control unit, puck or dongle type controller, remote control, or other dedicated controller configured for handheld operation. In some cases, the remote device 104 may be configured for placement on the user's person (e.g., as a bracelet or wristband, necklace or lanyard, etc.), attachment to the user's clothing (e.g., using Velcro or clips), or placement in another close location of the user (e.g., in a pocket, purse or other bag).

The remote device 104 may comprise one or more processors and a memory device (not shown) for storing instructions to be executed by the one or more processors. For example, the instructions may include software 120 that, when executed, cause the one or more processors to communicate with the wearable device 102, process data received from the wearable device 102, send audio signals and/or other data to the wearable device 102, and/or generate control signals for controlling operation of the wearable device 102 in order to carry out one or more of the techniques described herein. In various embodiments, the remote device 104 can be configured (e.g., using the software 120) to transmit one or more control signals to the wearable device 102 for controlling operation of the light source 112, the biometric sensor 116, and/or the audio output device 114. As another example, the remote device 104 may be configured (e.g., using the software 120) to send an audio signal to the wearable device 102 for playback by the audio output device 114. The remote device 104 may also be configured (e.g., using the software 120) to receive, from the wearable device 102, biometric data obtained by the biometric sensor 116 of the wearable device 102, process the received biometric data, and in some cases, depending on the biometric data, generate a new control signal for controlling operation of the light source 112.

The remote device 104 can be communicatively coupled to the wearable device 102 via a wired or wireless connection. In some embodiments, the remote device 104 comprises one or more wireless transceivers and other wireless communication circuitry (not shown) for creating a wireless connection with the wearable device 102, or more specifically, the wireless communication device 118 included therein. In other embodiments, the remote device 104 includes one or more communication ports for receiving a cable coupled to the wearable device 102. For example, the remote device 104 may include an audio output port for sending audio content to the wearable device 102 (such as, e.g., an audio signal for output by the audio output device 114), a data port for sending data content to the wearable device 102 (such as, e.g., control data for controlling operation of the light source 112) and/or receiving data from the wearable device 102 (such as, e.g., biometric data collected by the biometric sensor 116), and/or a combined audio and data port for sending and/or receiving both types of data.

FIG. 3 illustrates an exemplary wearable auricular light therapy device 200 configured to direct light therapy towards an auricular region of a wearer, or otherwise implement the light therapy techniques described herein, for example, like the wearable device 100 shown in FIG. 2, in accordance with embodiments. The wearable auricular light therapy device 200 (also referred to herein as “wearable device 200”) can be any type of headset or personal listening device comprising one or more ear cups 202 configured to be worn on or over, or otherwise coupled to, respective outer ears of a user (e.g., headphones, VR headset, etc.). FIG. 4 shows a front view of a single ear cup 202 (also referred to herein as “ear cup housing” or “housing”).

In various embodiments, the wearable device 200 may be headphones or earphones comprising a pair of ear cups 202 (also referred to herein as “a first ear cup and a second ear cup”) configured to be worn on either side of the user's head at the ears of the user. Each ear cup 202 may be configured to be worn on or over a respective outer ear 10 of the user. For example, in some cases, each ear cup 202 includes an ear pad 203 that is configured to rest on or against the corresponding outer ear 10. In other cases, the ear pad 203 in each ear cup 202 may be configured to entirely cover or cup the outer ear 10 when worn by the user. According to embodiments, the wearable device 200 may further comprise a headband or other band 204 connected to each ear cup 202 and configured to be worn on or adjacent to the user's head for securing the ear cups 202 to the user's ears.

In other embodiments, the wearable device 200 may be a headset comprising a single ear cup 202 configured to be worn on or over a select outer ear 10 of the user. In some cases, such wearable device 200 may also include the band 204 for securing the ear cup 202 to the user's head 20. In other cases, the ear cup 202 may be configured to be secured to the outer ear 10 using an ear clip or hook, instead of a headband (e.g., as shown in FIG. 7).

In some embodiments, the wearable device 200 may be a virtual reality (“VR”) headset that comprises one or more ear cups 202 configured to be positioned or worn over respective outer ears 10 of the user for implementing an audio aspect of a virtual reality experience. As will be appreciated, the VR headset may also include a visual component for implementing a visual aspect of the virtual reality experience and/or other components (e.g., haptic transducers, sensors, etc.) for implementing other sensory aspects of the experience.

Each ear cup 202 can be configured to implement the housing 106 by including all or a subset of the electronic components shown in FIG. 2. In embodiments where the wearable device 200 includes two ear cups 202, each of the first ear cup 202 and the second ear cup 202 may be identical in terms of content (e.g., include all of the same components), or may include different combinations of the components shown in FIG. 2. For example, as shown in FIGS. 3 and 4, each ear cup 202 can comprise an audio output device 206, which may be an audio speaker, loudspeaker, or other electroacoustic transducer for converting an audio signal into audible sound for transmission to the user's ear, like the audio output device 114 of FIG. 2. In some embodiments, each ear cup 202 may also comprise at least one light source 208 configured to direct a light output (e.g., light beam or light pulses) towards a select region of the outer ear 10 that is innervated by a vagus nerve of the user, similar to the light source 112 of FIG. 2. In addition, each ear cup 202 may comprise a biometric sensor 210 configured to detect or obtain biometric data of the user, such as, e.g., an electrical activity of the user's heart, like the biometric sensor 116 of FIG. 2. In other embodiments, only one of the ear cups 202 may include the light source 208 and/or the biometric sensor 210. Similarly, in some embodiments, one or more of the ear cups 202 may include a user interface 212 for receiving an input from the user, similar to the user interface 111 of FIG. 2. Moreover, though not shown, one or more of the ear cups 202 may include one or more of a processor, memory, and wireless communication device, like the processor 108, memory 110, and wireless communication device 118 of FIG. 2, respectively.

In some embodiments, each ear cup 202 may include two light sources 208 (e.g., two LEDs), for example, as shown in the illustrated embodiment. In other embodiments, each ear cup 202 may include more or fewer light sources 208 (e.g., only one light source 208 in each ear cup 202, etc.). In still other embodiments, the number of light sources 208 in each ear cup 202 may differ (e.g., one light source 208 in one ear cup 202 and two light sources 208 in the other ear cup 202, etc.). The exact number of light sources 208 placed in each ear cup 202 may be determined based on a number of factors, such as, for example, a location of the intended target area (or the area on the user that will receive the light output), a desired light field for the light output, power and/or space constraints, and more.

For example, in some embodiments, multiple light sources 208 are included in a given ear cup 202 in order direct each light source 208 towards a different innervated region of the outer ear 10. In one exemplary embodiment, a first light source 208 may be directed towards the concha area 12, while a second light source 208 may be directed towards the tragus area 14 or the ear lobe 16. In such cases, each light source 208 may be placed at a select location of the ear cup 202 and/or positioned at a different angle relative to the ear cup 202, or otherwise configured in order to target the corresponding area of the outer ear 10.

As another example, in some embodiments, the ear cup 202 may include two or more light sources 208 in order to accommodate users with different ear shapes and/or sizes. For example, the light sources 208 may be spaced apart relative to the audio output device 206 (e.g., as shown in FIGS. 3 and 4), so that one of the light sources 208 is more likely to be positioned at or near the concha area 12 of a larger sized outer ear 10 and the other light source 208 is more likely to be positioned at or near the concha area 12 of a smaller sized outer ear 10.

Given the variety of embodiments described herein, for ease of explanation, the following paragraphs use the singular term “the light source 208” to mean “one or more light sources 208” or the like, as will be appreciated.

As shown, each of the audio output device 206, the light source 208, and biometric sensor 210 can be positioned within or near a center of the ear cup 202 in order to have better or more direct access to the intended region of the outer ear 10 of the user for implementing the techniques described herein. For example, each ear cup 203 may have a generally annular shape with a central opening 211 configured to provide an open space for placement of the light source 208 and the audio output device 206. More specifically, the audio output device 206 may be positioned at or towards a center of the opening 211 in order to direct sound towards an ear canal (not shown) of the user.

Moreover, in various embodiments, the light source 208 can be positioned above the audio output device 206, so that a light output emitted by the light source 208 can be directed towards the concha area 12 of the outer ear 10 (e.g., as shown in FIG. 1A). In other embodiments, the light source 208 may be positioned in another location of the central opening 211 and/or the ear cup 202 that enables emission of the light output towards another area of the outer ear 10 that is innervated by an auricular branch of the vagus nerve (e.g., the tragus area 14 or the ear lobe area 16 shown in FIG. 1A).

In addition, the biometric sensor 210 can be positioned on the ear pad 203 just outside the central opening 211 in order to be adjacent to, or ensure sufficient contact with, a select surface, or skin surface, of the outer ear 10 that enables detection of biometric data. For example, the biometric sensor 210 may be configured to make contact with a skin surface adjacent to an artery that is anterior to the tragus area 14 of the outer ear 10 in order to detect a pulse of the user. In other embodiments, the biometric sensor 210 may be positioned in any other location of the ear cup 202 that enables the biometric sensor 210 to obtain appropriate biometric readings of the user. In some embodiments, the biometric sensor 210 may be positioned at an angle relative to the ear cup 202 and/or the central opening 211 in order to tilt the biometric sensor 210 towards the select surface of the outer ear 10 or otherwise ensure sufficient contact with that surface.

FIG. 5 shows a partial top view of the ear cups 202 positioned on either side of a head 20 of a user, so that the first ear cup 202 is adjacent to a first outer ear 10 of the user and the second ear cup 202 is adjacent to a second outer ear 10 of the user. Also in FIG. 5, the headband 204 and a top section of each ear cup 202 are removed to show an internal configuration of certain components.

More specifically, in various embodiments, the light source 208 can be configured to direct a light output 213 substantially perpendicular to a skin surface covering or adjacent to the target area of the outer ear 10 in order to maximize transmission of the light output 213 to the target area. For example, as shown in FIG. 5, an emitting end 214 of the light source 208 may be positioned at an angle θ relative to the ear cup 202 so that the light output 213 is pointed substantially perpendicular to the target area when the ear cup 202 is coupled to the outer ear 10. The exact value of the angle θ may vary depending on a shape, size, and/or other configuration of the ear cup 202, a location of the light source 208 within the ear cup 202, a size and/or type of light source 208, a location of the target area within the outer ear 10, a size of the target area, and/or other relevant factors. In one exemplary embodiment, the light source 208 is positioned at an angle of 35 degrees relative to the ear cup housing 202 in order to direct the light output 213 substantially perpendicular to a skin surface of the concha area 12 of the outer ear 10. In some embodiments, the angle θ of the light source 208 may be fixed, for example, at the time of manufacturing. In other embodiments, the angle θ of the light source 208 may be adjustable, for example, based on control signals received from the processor, as described herein. As shown in FIG. 5, the second ear cup 202 may be a mirror image of the first ear cup 202, so that the light source 208 included in each ear cup 202 is angled or titled towards the corresponding outer ear 10 from either side of the user's head 20.

FIGS. 6A through 6C illustrate another exemplary wearable light therapy device 300 configured to implement the light therapy techniques described herein, for example, as the wearable device 100 shown in FIG. 2, in accordance with embodiments. The wearable light therapy device 300 (also referred to herein as “wearable device 300”) can be any type of personal listening device comprising one or more earpieces 302 configured to be worn at least partially within, or otherwise coupled to, respective outer ears of a user (e.g., earphones, earbuds, air pods, in-ear headphones, headset, etc.). FIG. 6A shows a first side view of an exemplary earpiece 302 of the device 300, FIG. 6B shows a second side view of the same, opposite the first, and FIG. 6C shows a front view of the earpiece 302. FIG. 7 shows the earpiece 302 coupled to an outer ear 10 of a user, in accordance with embodiments.

In various embodiments, the wearable device 300 may be earphones, earbuds, air pods, in-ear headphones, or other personal listening device comprising a pair of earpieces 302 configured to be worn on either side of the user's head at the ears of the user. For example, the earpieces 302 may comprise a first earpiece 302 configured to be coupled to, or at least partially within, a first outer ear of the user and a second earpiece 302 configured to be coupled to, or at least partially within, a second outer ear of the user. In some cases, the wearable device 300 may further include a wire, cable, band, or other mechanism (not shown) for physically coupling the two earpieces 302 together, mechanically and/or electronically. In other cases, the earpieces 302 may be configured to wirelessly communicate with each other and/or the remote device 104 shown in FIG. 2.

In other embodiments, the wearable device 300 may be configured as a headset or other personal listening device having a single earpiece 302 configured for coupling to, or at least partially within, a select outer ear 10 of the user.

As shown, the earpiece 302 comprises an ear bud 303 configured to be inserted into and/or rest against a portion of the outer ear 10, such as, for example, the concha area 12 and/or the tragus area 14 (as shown in FIG. 1), for transmission of audible sound to an ear canal of the user. For example, in some cases, the ear bud 303 may include an ear tip (not shown) configured to be inserted at least partially into the outer ear 10 near the tragus area 14 and just outside the ear canal. In other cases, the ear bud 303 may be configured to be inserted at least partially into the cavity formed on the outer ear 10 by the concha area 12 or otherwise rest against the concha area 12. The ear bud 303 can be configured as a housing that includes, or houses, certain electrical components of the ear piece 302, as described herein.

The earpiece 302 also comprises an attachment component 304 for securing the ear bud 303 to the outer ear 10 or otherwise keeping the ear piece 302 in place on the outer ear 10. In the illustrated embodiment, the attachment component 304 is configured as an ear hook that extends up along a front of the outer ear 10, hooks over a top of the outer ear 10, and extends down along the back region 18 of the outer ear 10. In other embodiments, the attachment component 304 may be configured as a clip or other mechanism capable of coupling or attaching the ear bud 303 to the outer ear 10 of the user.

In various embodiments, the wearable device 300 may be similar to the wearable device 200 of FIGS. 3-5 and/or the wearable device 100 of FIG. 2. For example, like the ear cup 202, the earpiece 302 can be configured to implement the housing 106 by including all or a subset of the electronic components shown in FIG. 2. In particular, as shown in FIG. 6B, the earpiece 302, or more specifically, the ear bud 303 included therein, may include an audio output device 306 for converting an audio signal into audible sound to be transmitted to an ear canal of the user, like the audio output device 114 of FIG. 2 and/or the audio output device 306 shown in FIGS. 3 and 4.

In addition, the earpiece 302 may include a light source 308 (e.g., an LED or other light device) configured to direct a light output towards a target area of the outer ear 10, like the light source 112 of FIG. 2 and/or the light source 208 shown in FIGS. 3 and 4. In some embodiments, the light source 308 may be positioned at a preset angle relative to the ear bud 303 or otherwise configured to direct the light output substantially perpendicular to a skin surface of the target area of the outer ear 10, for example, like the light source 208 shown in FIG. 5. In some cases, the angle at which the light source 308 is positioned relative to the ear bud 303 may be adjustable, as described herein. The light source 308 is described in more detail below

Moreover, as also shown in FIG. 6B, the earpiece 302 may include a biometric sensor 310 for detecting or obtaining biometric data of the user, such as, e.g., an electrical activity of the user's heart, like the biometric sensor 116 of FIG. 2 and/or the biometric sensor 210 of FIGS. 3 and 4. According to various embodiments, the biometric sensor 310 can be configured to make sufficient contact with a select surface (e.g., skin surface) of or near the outer ear 10 to enable detection of the biometric data, as described in more detail below.

Also, as shown in FIG. 6A, the earpiece 302 may also include a user interface 312 for receiving an input from the user, similar to the user interface 111 of FIG. 2. Moreover, though not shown, the earpiece 302 may include one or more of a processor, memory, and wireless communication device, like the processor 108, memory 110, and wireless communication device 118 of FIG. 2, respectively. In some embodiments, the wireless communication device may be used to communicatively couple the first earpiece 302 and the second earpiece 302, in addition to wireless communicating with the remote device 104 of FIG. 2.

In embodiments with two earpieces 302, the first earpiece 302 and the second earpiece 302 may be identical in terms of content (e.g., include all of the components shown in FIGS. 6A to 6C), but mirror images of each other. In other cases, each of the earpieces 302 may include a different combination of components (e.g., a different subset of the components shown in FIGS. 6A to 6C). For example, the first earpiece 302 may include all of the components shown in FIGS. 6A to 6C, while the second earpiece 302 may include a smaller subset of those components.

As shown, in various embodiments, the earpiece 302 can include multiple light sources 308 (e.g., two LEDs, three LEDs, etc.) positioned at different locations of the ear bud 303 and/or the attachment component 304 in order to target different areas of the outer ear 10. For example, as shown in FIG. 6B, one or more first light sources 308a may be positioned on a front surface of the ear bud 303 in order to direct a first light output towards the concha region 12, tragus region 14, or other area at the front of the outer ear 10. In addition, as shown in FIG. 7, one or more second light sources 308b may be positioned on a rear surface of the attachment component 304 in order to direct a second light output towards the back surface 18 of the outer ear 10, the scalp area 19 near the outer ear 10, or other appropriate area at the back of the outer ear 10. For ease of explanation, the one or more first light sources 308a and the one or more second light sources 308b are collectively referred to herein as “the light source 308.”

As shown, the one or more second light sources 308b may include two light sources or elements (e.g., LEDs) arranged vertically, or in a line, along a bottom extension of the attachment component 304. In some cases, the second light sources 308b can be configured to increase a light field of the light output directed towards the back of the outer ear 10, for example, by turning both lights on simultaneously during a therapy session. In other cases, only one of the second light sources 308b may be selectively turned on, for example, depending on a size of the user's ear and/or head, as the exact location of the back surface 18 of the outer ear 10 and/or the scalp area 19 near the outer ear 10 may vary from user to user.

In some embodiments, the earpiece 302 includes multiple biometric sensors 310 positioned at different locations of the ear bud 303 and/or the attachment component 304 in order to obtain biometric data from different areas at or near the outer ear 10, as shown in FIGS. 6B and 7. For example, the earpiece 302 may include a front biometric sensor 310 located on the front surface of the ear bud 303 and configured to measure an electrical activity of the user's heart from the front of the outer ear 10. As shown in FIG. 6B, the front biometric sensor 310 may be positioned on the ear bud 303 so as to contact a skin surface near the tragus area 14, such as, e.g., the surface that is near an artery that runs anterior to the tragus area 14, like the biometric sensor 210 of FIGS. 3 and 4. The earpiece 302 may also include a back biometric sensor 310 located on the back surface of the attachment component 304 and configured to measure an electrical activity of the user's heart from or near the back of the outer ear 10. As shown in FIG. 7, the back biometric sensor 310 may be positioned on the attachment component 304 so as to contact a skin surface of the user's scalp that is near a back of the outer ear 10, such as, e.g., near the scalp area 19 or other surface that is adjacent an artery. In some embodiments, changes in certain biometric data (e.g., blood pressure) can be identified by comparing the biometric readings obtained by the sensors 310 at different locations of the user's body (such as, e.g., periodic pulse metering, heart rate, or blood flow measurements).

In other embodiments, the earpiece 302 includes either the front biometric sensor 310 or the back biometric sensor 310, instead of both. In some embodiments, the two earpieces 302 of the wearable device 300 may have biometric sensors 310 in two different locations, for example, in order to improve the quality of the biometric data by varying the data collection sites. As will be appreciated, the biometric sensor 310 may be positioned at any other location of the earpiece 302 that enables detection of biometric data (e.g., pulse rate, etc.) at or near the outer ear 10, in accordance with the techniques described herein.

FIG. 8 illustrates an example process or method 400 for providing light therapy at or near an outer ear of a user, in accordance with embodiments. The method 400 can be carried out by a computing device, such as, for example, the wearable light therapy device 102 shown in FIG. 2, the wearable device 200 shown in FIGS. 3-5, the wearable device 300 shown in FIGS. 6A-7, and/or the remote device 104 shown in FIG. 2. As an example, the method 400 may be implemented, at least in part, by at least one processor of the computing device (e.g., processor 108 shown in FIG. 2) executing a software application stored in a memory of the computing device (e.g., memory 110 shown in FIG. 2). Further, to carry out the operations of the method 400, the computing device can interact with one or more other devices communicatively coupled thereto, such as, for example, a remotely located computing device such as a server computer, a connected device (e.g., remote device 104 shown in FIG. 2), and/or other external device in wireless or wired communication with the computing device. In some embodiments, the method 400 may be carried out by a system (e.g., the light therapy system 100 of FIG. 2), or by one or more computing devices working together or in a distributed manner (e.g., the remote device 104 and the wearable device 102 of FIG. 2).

As shown in FIG. 8, the method 400 can begin at step 402 with starting a timer for a new light therapy session, in order to track a progress of the session and/or limit a length of the session to a select time period. For example, the at least one processor may be configured (e.g., using software) to send a control signal to the timer to turn on the timer and/or set the timer to the select time period. The select time period may be a predetermined length of time, such as, e.g., 20 minutes, or adjustable by the user, for example, using a user interface of the computing device (e.g., user interface 111 of FIG. 2). The timer may be implemented in hardware and/or software and may at least partially reside in the computing device. In various embodiments, the timer may be automatically controlled by the at least one processor. In some embodiments, the timer may be controlled in response to one or more inputs received via the user interface of the computing device. Once the timer is started at step 402, the timer may continue to run in the background, for example, until the time period ends or another event occurs, as described below.

The method 400 can also include, at step 404, playing an audible sound using an audio output device (e.g., audio output device 114 of FIG. 2). For example, the at least one processor may be configured to send an audio signal to the audio output device for playback or otherwise instruct the audio output device to generate an audible sound based on an audio signal. The audible sound may include music or musical content, human speech or speech content, or any combination thereof. In some cases, the audible sound may be an audio cue informing the user of an event (such as, e.g., the start or end of light therapy, etc.), or other audio content comprising instructions to the user during the therapy session (such as, e.g., telling the user to relax, etc.). In other cases, the audible sound may be relaxing music, audio content for EMDR, and/or other audio content that is played during or throughout the therapy session. The audio output device may be configured to continue playing audible sounds until a second control signal instructing the audio output device to stop playback of the sounds is received from the at least one processor, or until playback of the audio signal is complete.

The method 400 further includes, at step 406, turning on a biometric sensor (e.g., biometric sensor 116 of FIG. 2) to obtain biometric data of the user, or otherwise receiving biometric data of the user from the biometric sensor. For example, the at least one processor may be configured to send or transmit a control signal to the biometric sensor to turn on the biometric sensor and start collecting the biometric data of the user. The biometric sensor may also be instructed to send the obtained biometric data to the at least one processor for analysis, for example, in order to determine whether the biometric data indicates an abnormal user response (e.g., at step 410). The biometric data may include an electrical activity of a heart of the user (e.g., heart rate, heart rate variability, etc.) or any other type of biometric data. In various embodiments, the biometric sensor may be configured to collect an initial biometric data of the user and store the initial data in a memory (e.g., memory 110 of FIG. 2) as a baseline measure, or threshold value, for the user. The baseline value may be used to determine whether the user is experiencing a negative biometric response during the light therapy session (e.g., at step 410). The biometric sensor may be configured to continue obtaining biometric data until a second control signal instructing the biometric sensor to stop detecting biometric data is received from the at least one processor (e.g., at step 418).

At step 408, the method 400 includes turning on a light source (e.g., light source 112 of FIG. 2) to initiate emission of a light output by the light source towards a region of an outer ear of the user that is innervated by an auricular vagus nerve of the user. For example, the at least one processor may be configured to send a first control signal to the light source to turn on or otherwise begin emission of the light output. The light source may be configured to continue emitting the light output until a second control signal instructing the light source to stop the emission is received from the at least one processor (e.g., at step 416).

In various embodiments, the outputs or activities initiated at steps 404, 406, and/or 408 may continue to occur, simultaneously or correspondingly, for a duration of the method 400, such as, e.g., until the select time period ends (e.g., at step 412) or extenuating circumstances arise (e.g., at step 410). For example, the at least one processor may be configured to simultaneously receive biometric data of the user and provide the light output to the user by instructing the light source to emit the light output while the biometric sensor is obtaining the biometric data of the user. As another example, the at least one processor may be configured to simultaneously provide the light output and the audible sound to the user by instructing the light source to emit the light output while the audio output device is playing the audible sound. In some embodiments, the at least one processor may be configured to instruct the audio output device to generate the audible sound in association with the light output, for example, so that the sound corresponds with the light (e.g., an audible cue indicating the start or end of the light output, etc.).

In some embodiments, the control signals and/or audio signals associated with steps 404, 406, and/or 408 may be received at the wearable device from a remote device (e.g., remote device 104 of FIG. 2), for example, using a wireless connection (e.g., wireless communication device 118 of FIG. 2) or a wired connection. Such received signals may be provided to the at least one processor for distribution to the appropriate components of the wearable device, as described herein. The remote device may include software (e.g., software application 120) configured to generate the control signals and/or provide the audio signals for controlling operation of the wearable device. In some embodiments, the biometric data obtained by the biometric sensor may be sent, via the at least one processor, to the remote device for analysis, as described herein.

Step 410 includes determining whether the biometric data obtained for the user is indicative of an abnormal user response to the light therapy session, or otherwise determining an abnormal user response based on the received biometric data. For example, the at least one processor, or the software application stored in the remote device, may be configured to detect an abnormal or negative user response if the biometric data of the user falls below a predetermined threshold or baseline measure, and may be configured to determine a normal user response if the biometric data meets or exceeds the baseline measure. In embodiments where the biometric data is heart rate variability (“HRV”) data, an abnormal user response may be detected if the HRV readings of the user drop and stay below the baseline measure for a select length of time, while a normal user response may be detected if the HRV readings increase or remain the same over the select length of time, for example. In the illustrated embodiment, the at least one processor is configured to analyze the biometric data that is received while the light output is directed towards the user's outer ear. In other embodiments, the at least one processor may be configured to determine whether there is an abnormal user response before the light output is emitted, as further described below.

If a normal user response is detected at step 410 (e.g., “No”), the method 400 continues to step 412, which includes determining whether there is time left on the timer initiated at step 402. For example, the at least one processor may check whether the timer is still running. If there is time left (e.g., “Yes”), the method 400 continues to step 414, which includes continuing the light therapy session, for example, by continuing emission of the light output towards the user, collection of biometric data from the user, and playing audible sounds for the user. From step 414, the method 400 returns back to step 410 to check for an abnormal user response based on the new biometric data collected at step 414.

If an abnormal user response is detected at step 410 (e.g., “Yes”), the method 400 continues to step 416, which includes automatically turning off the light source or otherwise stopping transmission of the light output towards the user. For example, responsive to the abnormal user response determined at step 410, the at least one processor may be configured to transmit a second control signal to the light source, the second control signal configured to stop emission of the light output. In this manner, the light therapy session can be automatically ended upon determining that the user is responding negatively to the light therapy. The method 400 may also continue to step 416 from step 412 if there is no time left on the timer (e.g., “No”), i.e. once the select time period for the light therapy session has ended.

From step 416, the method 400 continues to step 418, which includes taking recovery measurements of the user using the biometric sensor. For example, the at least one processor may be configured to keep the biometric sensor on, or otherwise cause the biometric sensor to continue collecting biometric data of the user, after the light source is turned off. The recovery measurements may be used to determine whether the user is experiencing, or still experiencing, an abnormal biometric response after stopping emission of the light output. The recovery measurements may also be used to measure sustained elevated activity of the vagus nerve following a normal user response, which may be used to track or measure beneficial outcomes of the therapy session. The recovery measurements may be provided to the remote device for further analysis and/or storage in a memory for future retrieval. Once the recovery measurements are complete, the biometric sensor may be turned off, for example, in response to receiving a control signal from the at least one processor, and the method 400 may end.

Though not shown, in some embodiments, the method 400 can be configured to include a biofeedback loop that enables the at least one processor to adjust the light output, or one or more settings of the light source, based on, or responsive to, the biometric data collected by the biometric sensor. For example, if the at least one processor determines that the user's HRV readings are dropping towards, but have not yet crossed, the threshold, the at least one processor may be configured to stabilize or prevent a further decrease in the user response by automatically adjusting an intensity, duration, or other suitable parameter of the light output accordingly. In such cases, the biofeedback loop may continue until the select time period ends, stabilization of the user response is detected (e.g., the user's biometric data remains steady above the threshold for a predetermined amount of time), or an abnormal user response is detected (i.e. despite the automatic adjustments). Conversely, if the at least one processor detects no shift in HRV readings, the at least one processor may be configured to increase the intensity of light or activate more lights (either to the same area or other wavelengths for targeting other areas), until an HRV response is detected. Thus, the at least one processor can be configured to optimize, or “bio-tune,” the light therapy experience for each individual user, as needed. As an example, in embodiments where the wearable device is a VR headset, the bio-tuning process may be used to provide vagus nerve stimulation to help treat or minimize motion sickness experienced by a user while using the VR headset during a virtual reality session.

In some cases, the bio-tuning process may be performed prior to, or separately from, the method 400. For example, the bio-tuning process may start with receiving, from the biometric sensor, biometric data of a user (e.g., as in step 406) prior to turning on the light source, and may further include, determining an abnormal user response based on the received biometric data (e.g., as in step 410). From there, the bio-tuning process may continue with, responsive to the abnormal user response, transmitting, to the light source, a control signal configured to initiate emission of a light output towards a region at or near an outer ear of the user, the region innervated by a branch of a vagus nerve of the user. That is, the at least one processor may be configured to turn on the light source once an abnormal user response is detected, in order to use the light output to help alleviate the abnormal response (e.g., low heart rate activity). In some embodiments, the bio-tuning process may further include receiving, from the biometric sensor, additional biometric data of the user while the light output is directed towards the region; determining a normal user response based on the additional biometric data; and responsive to the normal user response, transmitting, to the light source, a second control signal configured to stop emission of the light output, or otherwise turn off the light source (e.g., as in step 416). The bio-tuning process may end once the user's biometric response is back to a normal or desirable level.

Thus, an improved light therapy device, system, and method are provided herein that can stimulate or activate a vagus nerve of the user using a light output directed towards an innervated region of the user, monitor the user's response to the light output, and automatically shut off the light output if a negative user response is detected, or use the light output to stabilize the user's biometric response if initially abnormal or negative. As an example, the targeted region may be an area of or near an outer ear of the user that is innervated by an auricular or periauricular branch of the vagus nerve, and the light therapy device may be configured to be worn on or near the outer ear of the user (e.g., earphones, headphones, headset, etc.). The innervated region may be the concha, tragus, or earlobe of the outer ear, a back of the outer ear, or a scalp area near the back of the outer ear, for example. In some cases, different colors of light may be used to target different innervated regions to maximize effectiveness.

In accordance with certain aspects, a wearable device is provided, the wearable device comprising: a housing configured for coupling to an outer ear of a user; a light source disposed in the housing and configured to direct a light output towards a region of the outer ear that is innervated by a branch of a vagus nerve of the user; and at least one processor disposed in the housing and communicatively coupled to the light source, the at least one processor configured to transmit, to the light source, a control signal for controlling emission of the light output by the light source. According to some aspects, said region of the outer ear includes a concha area of the outer ear and/or a scalp area near a back of the outer ear. According to some aspects, said region of the outer ear additionally or alternatively includes a tragus area of the outer ear and/or a back surface of the outer ear.

According to various aspects, the wearable device further comprises an audio output device disposed in the housing and communicatively coupled to the at least one processor, the audio output device configured to generate an audible sound based on an audio signal received from the at least one processor. According to some aspects, the wearable device further comprises a wireless communication device for enabling wireless communication with a remote device, the wireless communication device communicatively coupled to the at least one processor and configured to receive, from the remote device, the control signal for controlling emission of the light output and the audio signal for generating the audible sound. According to some aspects, the at least one processor is further configured to simultaneously provide the light output and the audible sound to the user. According to some aspects, the audible sound includes music and/or human speech.

In accordance with other aspects, a system is provided, the system comprising a first housing configured for coupling to a first outer ear of a user, the first housing comprising a first light source configured to direct a first light output towards a first region at or near the first outer ear, the first region innervated by a first branch of a vagus nerve of the user; a second housing configured for coupling to a second outer ear of the user, the second housing comprising a second light source configured to direct a second light output towards a second region at or near the second outer ear, the second region innervated by a second branch of the vagus nerve; and one or more processors configured to provide a first control signal to the first light source to control emission of the first light output, and a second control signal to the second light source to control emission of the second light output.

According to various aspects, the first housing further comprises a first audio output device configured to generate a first audible sound based on a first audio signal received from the one or more processors, and the second housing further comprises a second audio output device configured to generate a second audible sound based on a second audio signal received from the one or more processors. According to some aspects, the one or more processors are further configured to simultaneously provide each of the light outputs and each of the audible sounds to the user. According to some aspects, the system further comprises a wireless communication device configured to receive the first and second control signals and the first and second audio signals from a remote device.

In accordance with still other aspects, a method performed by at least one processor in communication with a light source and a biometric sensor is provided, the method comprising: transmitting, to the light source, a first control signal configured to initiate emission of a light output towards a region at or near an outer ear of a user, the region innervated by a branch of a vagus nerve of the user; receiving, from the biometric sensor, biometric data of the user while the light output is directed towards the region; determining an abnormal user response based on the received biometric data; and responsive to the abnormal user response, transmitting, to the light source, a second control signal configured to stop emission of the light output. According to various aspects, the method further comprises transmitting, to an audio output device in communication with the at least one processor, an audio signal configured to generate an audible sound in association with the light output.

In embodiments, the components of the light therapy system 100, the wearable light therapy device 200, and/or the wearable light therapy device 300 may be implemented in hardware (e.g., discrete logic circuits, application specific integrated circuits (ASIC), programmable gate arrays (PGA), field programmable gate arrays (FPGA), digital signal processors (DSP), microprocessor, etc.), using software executable by one or more computers, such as a computing device having a processor and memory (e.g., a personal computer (PC), a laptop, a tablet, a mobile device, a smart device, thin client, etc.), or through a combination of both hardware and software. For example, some or all components of the light therapy system 100 and/or any of the devices 200 and 300 may be implemented using discrete circuitry devices and/or using one or more processors (e.g., audio processor and/or digital signal processor) executing program code stored in a memory, the program code being configured to carry out one or more processes or operations described herein, such as, for example, the method shown in FIG. 8. Thus, in embodiments, the system 100 and/or any of the devices 200 and 300 may include one or more processors, memory devices, computing devices, and/or other hardware components not shown in the figures.

All or portions of the processes described herein, including method 400 of FIG. 8 may be performed by one or more processing devices or processors (e.g., analog to digital converters, encryption chips, etc.) that are within or external to the corresponding system (e.g., system 100 of FIG. 2). In addition, one or more other types of components (e.g., memory, input and/or output devices, transmitters, receivers, buffers, drivers, discrete components, logic circuits, etc.) may also be used in conjunction with the processors and/or other processing components to perform any, some, or all of the steps of the method 400. As an example, in some embodiments, the method described herein may be carried out by a processor (e.g., processor 108 of FIG. 2) executing software stored in a memory (e.g., memory 110 of FIG. 2). The software may include, for example, program code or computer program modules comprising software instructions executable by the processor. In some embodiments, the program code may be a computer program stored on a non-transitory computer readable medium that is executable by a processor of the relevant device.

The terms “non-transitory computer-readable medium” and “computer-readable medium” include a single medium or multiple media, such as a centralized or distributed database, and/or associated caches and servers that store one or more sets of instructions. Further, the terms “non-transitory computer-readable medium” and “computer-readable medium” include any tangible medium that is capable of storing, encoding or carrying a set of instructions for execution by a processor or that cause a system to perform any one or more of the methods or operations disclosed herein. As used herein, the term “computer readable medium” is expressly defined to include any type of computer readable storage device and/or storage disk and to exclude propagating signals.

Any process descriptions or blocks in figures should be understood as representing modules, segments, or portions of code which include one or more executable instructions for implementing specific logical functions or steps in the process, and alternate implementations are included within the scope of the embodiments of the invention in which functions may be executed out of order from that shown or discussed, including substantially concurrently or in reverse order, depending on the functionality involved, as would be understood by those having ordinary skill in the art.

It should be understood that examples disclosed herein may refer to computing devices and/or systems having components that may or may not be physically located in proximity to each other. Certain embodiments may take the form of cloud based systems or devices, and the term “computing device” should be understood to include distributed systems and devices (such as those based on the cloud), as well as software, firmware, and other components configured to carry out one or more of the functions described herein. Further, as noted above, one or more features of the system may be standalone or physically remote (e.g., remote device 104 of FIG. 2) and may be communicatively coupled to the computing device.

It should be noted that in the description and drawings, like or substantially similar elements may be labeled with the same reference numerals. However, sometimes these elements may be labeled with differing numbers, such as, for example, in cases where such labeling facilitates a more clear description. Additionally, the drawings set forth herein are not necessarily drawn to scale, and in some instances proportions may have been exaggerated to more clearly depict certain features. Such labeling and drawing practices do not necessarily implicate an underlying substantive purpose. As stated above, the specification is intended to be taken as a whole and interpreted in accordance with the principles of the invention as taught herein and understood to one of ordinary skill in the art.

In this disclosure, the use of the disjunctive is intended to include the conjunctive. The use of definite or indefinite articles is not intended to indicate cardinality. In particular, a reference to “the” object or “a” and “an” object is intended to also denote one of a possible plurality of such objects.

This disclosure describes, illustrates and exemplifies one or more particular embodiments of the invention in accordance with its principles. The disclosure is intended to explain how to fashion and use various embodiments in accordance with the technology rather than to limit the true, intended, and fair scope and spirit thereof. That is, the foregoing description is not intended to be exhaustive or to be limited to the precise forms disclosed herein, but rather to explain and teach the principles of the invention in such a way as to enable one of ordinary skill in the art to understand these principles and, with that understanding, be able to apply them to practice not only the embodiments described herein, but also other embodiments that may come to mind in accordance with these principles. The embodiment(s) provided herein were chosen and described to provide the best illustration of the principle of the described technology and its practical application, and to enable one of ordinary skill in the art to utilize the technology in various embodiments and with various modifications as are suited to the particular use contemplated. All such modifications and variations are within the scope of the embodiments as determined by the appended claims, as may be amended during the pendency of this application for patent, and all equivalents thereof, when interpreted in accordance with the breadth to which they are fairly, legally and equitably entitled.

What is claimed is:

1. A wearable device, comprising:
a housing configured for coupling to an outer ear of a user;
a light source disposed in the housing and configured to direct a light output towards a region of the outer ear that is innervated by a branch of a vagus nerve of the user; and
at least one processor disposed in the housing and communicatively coupled to the light source, the at least one processor configured to transmit, to the light source, a control signal for controlling emission of the light output by the light source.

2. The wearable device of claim 1, wherein the light source is configured to emit light at a wavelength selected from a range of about 420 nanometers to about 470 nanometers.

3. The wearable device of claim 1, wherein the light source is configured to emit light at a wavelength selected from a range of about 500 nanometers to about 565 nanometers.

4. The wearable device of claim 1, wherein the light source is configured to direct the light output substantially perpendicular to the region of the outer ear.

5. The wearable device of claim 1, wherein the region includes a concha area of the outer ear.

6. The wearable device of claim 1, wherein the region includes a scalp area near a back of the outer ear.

7. The wearable device of claim 1, further comprising a biometric sensor disposed within the housing and communicatively coupled to the at least one processor, wherein the biometric sensor is configured to: obtain biometric data of the user, and transmit the biometric data to the at least one processor, and the at least processor is further configured to control emission of the light output based on the received biometric data.

8. The wearable device of claim 7, wherein:
the control signal is configured to initiate emission of the light output;
the biometric sensor is further configured to: obtain the biometric data of the user during emission of the light output towards the user, and transmit the biometric data to the at least one processor; and
the at least one processor is further configured to: determine an abnormal user response based on the received biometric data, and responsive to the abnormal user response, transmit a second control signal to the light source, the second control signal configured to stop emission of the light output.

9. The wearable device of claim 7, wherein:
the biometric sensor is further configured to obtain the biometric data of the user prior to emission of the light output by the light source; and
the at least one processor is further configured to: determine an abnormal user response based on the received biometric data, and responsive to the abnormal user response, transmit the control signal to the light source, the control signal configured to initiate emission of the light output towards the region of the outer ear of the user.

10. The wearable device of claim 7, wherein the biometric data includes information associated with an electrical activity of a heart of the user.

11. The wearable device of claim 1, wherein the housing is an ear cup configured to be worn on or over the outer ear of the user.

12. The wearable device of claim 1, wherein the housing is an earpiece configured to be worn at least partially within the outer ear of the user.

13. The wearable device of claim 1, wherein the light source comprises at least one light emitting diode (“LED”).

14. A system, comprising:
a first housing configured for coupling to a first outer ear of a user, the first housing comprising a first light source configured to direct a first light output towards a first region at or near the first outer ear, the first region innervated by a first branch of a vagus nerve of the user;
a second housing configured for coupling to a second outer ear of the user, the second housing comprising a second light source configured to direct a second light output towards a second region at or near the second outer ear, the second region innervated by a second branch of the vagus nerve; and
one or more processors configured to provide a first control signal to the first light source to control emission of the first light output, and a second control signal to the second light source to control emission of the second light output.

15. The system of claim 14, further comprising a band connected to the first housing and the second housing, the band configured to be worn on or adjacent to a head of the user.

16. A method performed by at least one processor in communication with a light source and a biometric sensor, the method comprising:
transmitting, to the light source, a first control signal configured to initiate emission of a light output towards a region at or near an outer ear of a user, the region innervated by a branch of a vagus nerve of the user;
receiving, from the biometric sensor, biometric data of the user while the light output is directed towards the region;
determining an abnormal user response based on the received biometric data; and
responsive to the abnormal user response, transmitting, to the light source, a second control signal configured to stop emission of the light output.

17. The method of claim 16, wherein the biometric data includes information associated with an electrical activity of a heart of the user.

18. A method performed by at least one processor in communication with a light source and a biometric sensor, the method comprising:
receiving, from the biometric sensor, biometric data of a user;
determining an abnormal user response based on the received biometric data; and
responsive to the abnormal user response, transmitting, to the light source, a control signal configured to initiate emission of a light output towards a region at or near an outer ear of the user, the region innervated by a branch of a vagus nerve of the user.

19. The method of claim 18, further comprising:
receiving, from the biometric sensor, additional biometric data of the user while the light output is directed towards the region;
determining a normal user response based on the additional biometric data; and
responsive to the normal user response, transmitting, to the light source, a second control signal configured to stop emission of the light output.

20. The method of claim 28, wherein the biometric data includes information associated with an electrical activity of a heart of the user.
